(19) United States
(12) Patent Application Publication
(10) Pub. No.: US 2006/0122583 A1
(43) Pub. Date: Jun. 8, 2006

(54) Method and apparatus for performing myocardial revascularization

(75) Inventors: Benny Pesach, Rosh-Ha'ayin, IL (US); Michal Balberg, Jerusalem, IL (US)

Correspondence Address: WOLF, BLOCK, SCHORR & SOLIS-COHEN LLP, 250 PARK AVENUE, NEW YORK, NY, 10177, US

(73) Assignee: Glucon Inc, Boulder, CO (US)

(21) Appl. No.: 10/519,023

(22) PCT Filed: Jun. 25, 2003
(86) PCT No.: PCT/IL03/00534

§ 371 Date: Jan. 30, 2006

(30) Foreign Application Priority Data
Jun. 25, 2002 (US) 60391037

(51) Int. Cl. A61B 18/18

(52) U.S. Cl. 606/7; 606/12; 606/15

(57) ABSTRACT

Apparatus for forming a hole in a region of the heart muscle wall of a patient undergoing myocardial revascularization comprising: means for removing tissue from the region to form the hole; a light source that illuminates the region with light that generates photoacoustic waves therein; at least one acoustic sensor that generates signals responsive to the photoacoustic waves; and a controller that receives the signals and processes them to determine a characteristic of the region useable to control the means for removing tissue.

### RELATED APPLICATION

This application claims the benefit under 119(e) of 60/391,037 filed Jun. 25, 2002, the disclosure of which is incorporated herein by reference.

### FIELD OF THE INVENTION

The invention relates to methods and apparatus for removing tissue from a region of heart muscle to cause revascularization of the muscle.

### BACKGROUND OF THE INVENTION

In transmyocardial revascularization (TMR) and percutaneous myocardial revascularization (PMR) holes are created in heart muscle to stimulate angiogenesis in ischemic heart tissue. TMR and PMR are collectively referred to herein as a “myocardial revascularization” (MR).

In TMR a patient's chest is opened so that a surgeon can access the heart and “drill” holes, hereinafter “angiogenesis holes”, completely through the heart muscle wall from outside the heart, through the heart wall and into an inside chamber, generally the left ventricle, of the heart. Typically, the holes have diameters of about a millimeter and may be drilled mechanically or by ablating heart tissue by concentrating energy on the tissue to remove it and form the holes. Various forms of energy, such as for example electrical, RF and optical energy have been used to ablate heart tissue. After drilling, the surgeon prevents hemorrhaging and unwanted blood seepage from the inside of the heart into the chest cavity by applying pressure to the holes. In response to the applied pressure, ends of the holes near the outside of the heart seal sufficiently and relatively rapidly to prevent potentially damaging hemorrhaging.

In PMR a patient's chest is not opened and holes are drilled in the patient's heart from inside a heart chamber, generally the left ventricle, towards the outside of the heart using a catheter. The catheter has a first end, hereinafter a “drill end”, which is inserted into a suitable blood vessel, generally the femoral artery in the patient's groin, and threaded through the vascular system into the heart chamber. The drill end is positioned so that it contacts, or is in close proximity to, a region of heart muscle in which it is desired to drill a hole. A suitable form of ablative energy is input into a second end, hereinafter a “control end”, of the catheter located outside of the patient's body. The ablative energy is transported via an appropriate conduit in the catheter from the control end to the drill end. The transported energy is transmitted from the drill end to the desired region of the heart muscle to ablate tissue in the heart muscle and drill the hole.

Unlike in TMR, in PMR the surgeon does not have direct physical access to the drilled holes. As a result, in PMR, drilling must usually be more carefully controlled so that drilled holes do not perforate the heart muscle wall and lead to uncontrolled hemorrhaging into the chest cavity. Whereas care must be taken so that the drilled holes do not penetrate through the heart wall, the holes generally must be made sufficiently deep so that they are effective in stimulating angiogenesis. While it is not known to precisely how deep an angiogenesis hole should be in order for it to be effective in promoting angiogenesis, it appears that shallow holes are less effective in promoting angiogenesis than relatively deep holes.

U.S. Pat. No. 5,893,848 describes a PMR catheter for creating angiogenesis holes in heart tissue, the catheter having a stop that prevents the drill end of the catheter from penetrating into the heart tissue beyond a predetermined depth. The limit on the penetration depth prevents drilling holes in the heart tissue that are too deep and might penetrate through the heart wall. The patent also describes monitoring penetration depth of the drill end using energy, such as optical or acoustic energy transmitted from a suitable energy transmitter comprised in the catheter. Detectors positioned along the length of the catheter sense the transmitted energy. Detectors on the catheter that are located inside an angiogenesis hole being drilled by the catheter respond differently to the transmitted energy than detectors on the catheter that are outside the hole. The difference in the response is used to determine how deep the drill end has penetrated the heart tissue.

U.S. Pat. No. 6,200,310 describes monitoring PMR to determine whether angiogenesis holes generated in a region of a patient's heart using a catheter are effective in stimulating angiogenesis by monitoring an electrocardiogram of the region. The patent also describes transmitting ultrasound waves from the drill end of a catheter used in PMR to generate an ultrasound map of an angiogenesis hole that provides the dimensions, location and orientation of the hole. US Patent Application Publication 2001/0027316 A1, describes measuring thickness of tissue being drilled during myocardial revascularization using optical coherence reflectance or optical coherence tomography.

U.S. Pat. No. 6,024,703, describes a catheter used for ablative drilling with laser light of an angiogenesis hole in a region of the heart wall of a patient undergoing a TMR or PMR procedure. The laser light is delivered to a drill end of the catheter by an optic fiber and is transmitted to the heart wall region from an output end of the fiber. The drill end comprises an acoustic transducer. During drilling of an angiogenesis hole in the heart tissue region, the acoustic transducer is controlled to transmit acoustic waves that are incident on the region. Reflections of the transmitted ultrasound are used to determine depth of the hole, thickness and changes therein of the heart wall between the bottom of the hole and the epicardial surface of the heart and position of the output end of the optic fiber relative to the drill end. The information provided by the reflected ultrasound is used to control drilling of the hole. The disclosures of all the above referenced US Patents and Patent Application Publication are incorporated herein be reference.

An article by F. W. Cross et al., “Time-Resolved Photoacoustic Studies of Vascular Tissue Ablation at Three Wavelengths”, Appl. Phys. Lett. 50 (15) 13 Apr. 1987, pages 1019-1021, the disclosure of which is incorporated herein by reference, discusses ablation of normal and atheroma vascular tissue using laser light. The article describes “the application of fast time response acoustic transducers to study subthreshold thermoelastic and ablative response of normal and atheromatous human cadaver aorta subjected to UV and visible laser radiation”. Laser fluence thresholds at which a photoacoustic affect of laser light on the tissue becomes ablative is identified for the three wavelengths from differences in characteristics of acoustic pulses generated by the tissue responsive to laser fluence below and above threshold. Rate of tissue ablation is given as a function of fluence for the three wavelengths.

An article by S. Sato et al, “Nanosecond, High Intensity Pulsed Laser Ablation of Myocardium Tissue at the Ultraviolet, Visible, and Near-Infrared Wavelengths: In-Vitro Study”, Lasers in Surgery and Medicine 29:464-473 (2001) describes efficiency and characteristics of laser ablation for forming holes in myocardial tissue as a function of wavelength. Optical and acoustic emissions of the ablated tissue were used to study the ablation process. The article is incorporated herein by reference.

### SUMMARY OF THE INVENTION

An aspect of some embodiments of the present invention relates to providing apparatus for drilling angiogenesis holes in a myocardial revascularization (MR) procedure.

An aspect of some embodiments of the present invention relates to providing apparatus and a method for determining thickness of a region of heart muscle wall of a patient's heart in which a hole is drilled during myocardial revascularization.

An aspect of some embodiments of the present invention relates to providing apparatus and a method for determining a depth to which a hole is drilled in cardiac tissue during myocardial revascularization.

An aspect of some embodiments of the present invention relates to providing apparatus and a method for determining viability of cardiac tissue in which angiogenesis holes are drilled.

An aspect of some embodiments of the present invention relates to providing apparatus and a method for monitoring changes in cardiac tissue in a region of the heart in which holes are drilled during myocardial revascularization.

An aspect of some embodiments of the present invention relates to providing apparatus and a method for controlling formation of angiogenesis holes in cardiac tissue that are drilled by ablation during an MR procedure.

A myocardial revascularization apparatus (MRA), in accordance with an embodiment of the present invention, comprises means for removing heart tissue to form angiogenesis holes in a region of the heart and a light source for illuminating the region with light that generates sound waves in the region by the photoacoustic effect. The MRA comprises at least one acoustic sensor that generates signals responsive to the photoacoustic sound waves. A controller controls the light source and receives the signals generated by the at least one sensor. The controller processes the received signals to determine a characteristic of the photoacoustic waves and monitors and/or controls formation of the angiogenesis holes responsive to the determined characteristic.

In accordance with an embodiment of the present invention, to determine depth of a hole drilled by the MRA and thickness of a region of the heart wall of a patient in which the hole is drilled, the controller controls the light source to illuminate the region with at least one pulse of light that stimulates photoacoustic waves in the region. Photoacoustic waves stimulated by the light that are incident on the at least one sensor arrive at the at least one acoustic sensor at times that are functions of locations in the illuminated region at which they are generated. In accordance with an embodiment of the present invention, signals produced by the at least one acoustic sensor responsive to the incident photoacoustic waves are processed to determine spatial coordinates of the locations. The determined coordinates are used to determine a depth of the drilled hole and thickness of the heart wall region. Coordinates of the locations may be determined using methods known in the art or methods described in PCT Application WO 02/15776, the disclosure of which is incorporated herein by reference.

In accordance with an embodiment of the present invention, to determine viability of heart tissue and locate an ischemic region of the heart that is a suitable candidate for MR the MRA performs an assay of at least one analyte in the region that is indicative of a degree of ischemia. Among analytes that are indicative of ischemia and may be assayed in accordance with an embodiment of the present invention are for example oxygenated hemoglobin, cytochrome aa₃ redox or Hydrogen ions (corresponding to tissue pH).

To perform the assay, the controller controls the light source to illuminate the region with a pulse of light that is absorbed by the analyte and, as a result of absorption by the analyte, stimulates generation of photoacoustic waves in the region. Signals produced by the at least one sensor responsive to the photoacoustic waves are processed using methods known in the art or methods described in the above referenced PCT application to determine an absorption coefficient and/or scattering for the substance and therefrom a concentration of the substance in the region.

In some embodiments of the present invention, the assay is periodically repeated during the MR procedure to monitor changes in the analyte concentration and thereby changes in the tissue of the region. Apparatus and methods of determining tissue viability are discussed in a PCT application entitled “Method And Apparatus for Determining Tissue Viability” filed on even date with the present application, the disclosure of which is incorporated herein by reference.

In some embodiments of the present invention, an MRA drills angiogenesis holes by ablating heart tissue with a suitable ablative energy. Optionally, the ablative energy is optical energy. Ablative energy, in addition to removing tissue from a region of the heart to form a hole therein, can cause peripheral damage to tissue in a neighborhood of the hole that is formed. In some embodiments of the present invention, the MRA monitors peripheral damage to the tissue by monitoring response of the tissue to light that generates photoacoustic waves therein.

For example, as reported in U.S. Pat. No. 6,309,352, the disclosure of which is incorporated herein by reference, coagulated tissue generally exhibits a substantially different photoacoustic response to light than does non-coagulated tissue. By monitoring photoacoustic response to light of cardiac tissue in which a hole is drilled by ablation, in accordance with an embodiment of the present invention, possible coagulation damage to the tissue in a neighborhood of the hole is monitored.

In ablative drilling of holes in a region of the heart wall, vaporization of heart tissue by ablative energy generates thermoacoustic shock waves in the heart wall. In accordance with an embodiment of the present invention, the at least one acoustic sensor senses the shock waves and generates signals responsive thereto. The controller processes the signals to determine a characteristic of the shock waves, such as amplitude or integrated amplitude of the shock waves, to measure a rate of ablation of the heart tissue. The intensity of the ablative energy and/or its time dependence, i.e. pulse shape and pulse repetition frequency, is optionally controlled responsive to the determined characteristic.

It is noted that an MRA, which utilizes the photoacoustic effect, in accordance with an embodiment of the present invention, provides with a single device many different functions that are advantageous for performance of MR. An MRA, in accordance with an embodiment of the present invention, not only provides spatial mensuration for monitoring MR, but also different and varied measures of tissue viability and measures of tissue damage that might result from an MR procedure. It is also noted that many of these functions can be performed in real time, immediately prior to and during a same MR procedure.

An MRA in accordance with an embodiment of the present invention may be configured to perform TMR or PMR. For both TMR and PMR procedures, the at least one acoustic sensor may comprises at least one acoustic sensor located on the skin of a suitable region, such as the chest, of the person undergoing the procedure. When configured for performing PMR, components of the MRA are packaged in a suitable catheter, using any of various methods known in the art.

Whereas the above discussion refers to methods and apparatus for drilling holes in cardiac tissue, the methods and apparatus are not restricted to drilling holes in cardiac tissue. The methods and apparatus may be applied, with suitable modifications as might be required and would readily occur to a person of the art, to the formation of incisions in cardiac tissue other than holes and to holes or incisions different from holes in tissue other than cardiac tissue.

There is therefore provided in accordance with an embodiment of the present invention apparatus for forming a hole in a region of the heart muscle wall of a patient undergoing myocardial revascularization comprising: means for removing tissue from the region to form the hole; a light source that illuminates the region with light that generates photoacoustic waves therein; at least one acoustic sensor that generates signals responsive to the photoacoustic waves; and a controller that receives the signals and processes them to determine a characteristic of the region useable to control the means for removing tissue.

Optionally, the light source illuminates the region with at least one pulse of light at a wavelength at which light is absorbed by a substance in the region whose concentration can be used to assess a degree of ischemia in the region and wherein the controller processes the signals provided by the at least one acoustic sensor to assay the substance. Optionally, the substance is hemoglobin. Optionally, the hemoglobin is oxygenated. Additionally or alternatively, the substance is cytochrome aa₃ redox.

In some embodiments of the present invention, the light source illuminates the region with at least one pulse of light at a wavelength at which light is absorbed by water and determines temperature of the region responsive to the signals. Optionally the apparatus comprises a heat pump that generates a temperature difference between tissue in the region and an ambient temperature of the heart wall and wherein the controller thereafter determines temperature of the tissue as a function of time to assess a degree of ischemia in the region.

In some embodiments of the present invention, the light source illuminates the region with at least one light pulse prior to forming the hole and the controller processes the signals to determine a thickness of the heart wall in the region.

In some embodiments of the present invention, after onset of formation of the hole the light source illuminates the region with at least one light pulse that illuminates the bottom of the hole and the controller uses the signals generated by the at least one acoustic sensor to determine a depth for the hole. Optionally, the controller controls the means for removing tissue from the region responsive to the determined depth and stops formation of the hole by the means for removing tissue when a desired hole depth is reached.

In some embodiments of the present invention, the hole is formed in a first surface of the heart wall and deepened towards a second surface of the heart wall and during formation of the hole the light source illuminates the region with at least one light pulse that illuminates the bottom of the hole and the controller uses the signals generated by the at least one acoustic sensor to determine a thickness of the heart muscle wall between the bottom of the hole and the second surface. Optionally, the first surface is an inner surface of the heart wall. Optionally, the first surface is an outer surface of the heart wall.

In some embodiments of the present invention, the controller controls the means for removing tissue from the region responsive to the determined thickness and stops formation of the hole by the means for removing tissue when a desired thickness is reached.

In some embodiments of the present invention, the means for removing tissue comprises a source of ablative energy having an output port from which the ablative energy source provides energy for removing heart tissue by ablation. Optionally, the source of ablative energy illuminates the region with at least one pulse of ablative energy to form the hole. Optionally, the at least one ablative pulse generates an acoustic shock wave in the region responsive to which the at least one acoustic sensor generates signals that are transmitted to the controller and wherein the controller processes the signals to determine at least one characteristic of the shock waves. Optionally, the controller controls at least one characteristic of the at least one ablative pulse responsive to the determined at least one characteristic of the shock wave. At least one characteristic of the at least one ablative pulse is optionally at least one of pulse width, rise time, fall time, peak, and energy and repetition rate of the at least one ablative pulse. Additionally or alternatively, the at least one characteristic of the shock wave is at least one of temporal profile, duration, maximum pressure, minimum pressure, average pressure average intensity and integrated intensity of the acoustic shock wave.

In some embodiments of the present invention, the pulse generates an acoustic shock wave and wherein an acoustic sensor of the at least one acoustic sensor generates signals responsive to reflections of acoustic energy from the shock wave which the controller processes to determine a characteristic of the region. Optionally, the characteristic comprises a depth of the hole. Additionally or alternatively, the characteristic comprises a thickness of the heart muscle wall between the bottom of the hole and a surface of the wall.

In some embodiments of the present invention, the at least one acoustic sensor generates signals responsive to an acoustic shock wave generated by the at least one ablative pulse and the controller processes the signals to determine location of the source of the shock waves.

In some embodiments of the present invention, the at least one ablative pulse comprises a plurality of ablative pulses.

In some embodiments of the present invention, the light source illuminates the region with at least one pulse of light after onset of ablation and the controller uses signals generated by the at least one acoustic sensor responsive to photoacoustic waves to assess damage to tissue in the region of the hole caused by ablation. Optionally, the wavelength of the at least one light pulse is determined so as to increase a difference in the photoacoustic response of damaged tissue relative to undamaged tissue. Optionally, the damage comprises thermal damage. Optionally, the damage comprises acidosis.

In some embodiments of the present invention, the controller controls at least one characteristic of the ablative pulses responsive to the determined damage.

In some embodiments of the present invention, the controller processes the signals from the at least one acoustic sensor to determine a distance of the ablative energy output port to the bottom of the hole.

In some embodiments of the present invention, the ablative energy comprises electromagnetic energy.

In some embodiments of the present invention, the ablative energy comprises acoustic energy.

In some embodiments of the present invention, the ablative energy comprises optical energy.

In some embodiments of the present invention, the apparatus comprises a catheter having a drill end that is positioned in a neighborhood of or in contact with the region in order to form the hole and wherein the optical output aperture, the ablative energy output port and an acoustic sensor of the at least one acoustic sensor are mounted inside the catheter in a neighborhood of the drill end.

In some embodiments of the present invention, the controller processes signals that it receives from the at least one acoustic sensor to determine a location of the ablative energy output port.

In some embodiments of the present invention, the apparatus comprises a catheter having a drill end that is positioned in a neighborhood of or in contact with the region in order to form the hole and wherein the optical output aperture and an acoustic sensor of the at least one acoustic sensor are mounted inside the catheter in a neighborhood of the drill end.

In some embodiments of the present invention, the catheter is configured to perform percutaneous myocardial revascularization.

In some embodiments of the present invention, the catheter is configured to perform transmyocardial revascularization.

In some embodiments of the present invention, the at least one acoustic sensor comprises an external acoustic sensor coupled to the patient's skin.

In some embodiments of the present invention, the at least one acoustic sensor comprises an acoustic sensor of an ultrasonic imaging device.

There is further provided in accordance with an embodiment of the present invention, apparatus for forming a hole in a region of the heart muscle wall of a patient undergoing myocardial revascularization comprising: means for removing tissue from the region to form the hole; a light source that illuminates the region with light; an optical sensor that generates signals responsive to light from the light source that is reflected by the region; and a controller that receives the signals and processes them to determine at least one characteristic of the region useable to control the means for removing tissue.

Optionally, the characteristic is a substance indicative of viability of tissue in the region. Optionally, the substance is hemoglobin. Optionally, the hemoglobin is oxygenated. Optionally, the substance is cytochrome aa₃ redox. Optionally, the substance is Hydrogen ions.

### BRIEF DESCRIPTION OF FIGURES

Non-limiting examples of embodiments of the present invention are described below with reference to figures attached hereto and listed below. In the figures, identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1A schematically shows an MRA performing PMR on a region of heart tissue in accordance with an embodiment of the present invention;

FIG. 1B shows an enlarged view of the region shown in FIG. 1A undergoing PMR in accordance with an embodiment of the present invention;

FIG. 2 shows a schematic graph of pressure of photoacoustic waves stimulated in the region shown in FIGS. 1A and 1B, in accordance with an embodiment of the present invention;

FIG. 3A schematically shows an angiogenesis hole drilled in the region shown in FIGS. 1A and 1B, in accordance with an embodiment of the present invention;

FIG. 3B shows a schematic graph of pressure of photoacoustic waves stimulated during formation of the angiogenesis hole shown in FIG. 3A, in accordance with an embodiment of the present invention; and

FIG. 4 schematically shows sensing an acoustic shock wave generated by an ablative light pulse used to form the hole shown in FIG. 3A, in accordance with an embodiment of the present invention.

### DETAILED DESCRIPTION OF EXEMPLARY EMBODIMENTS

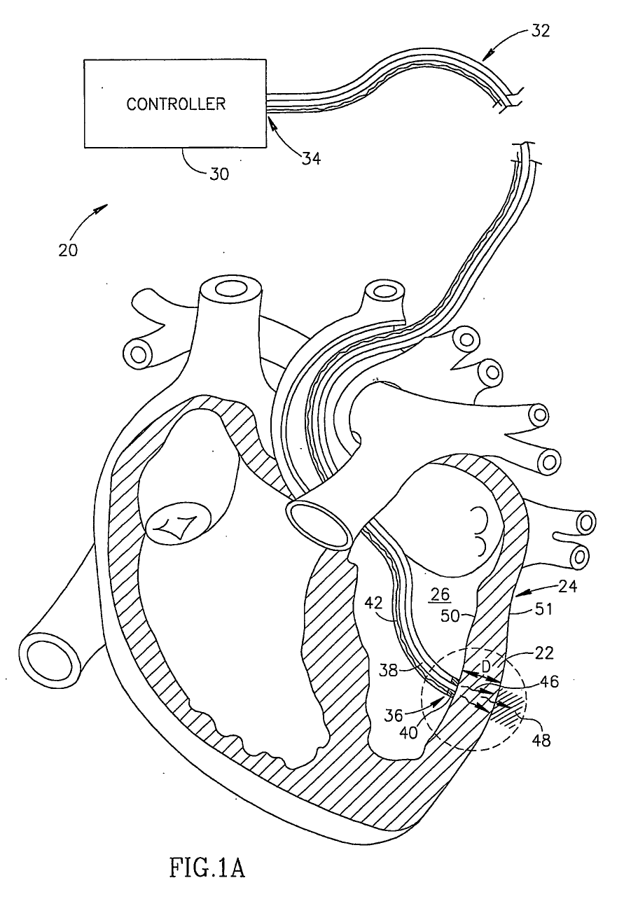

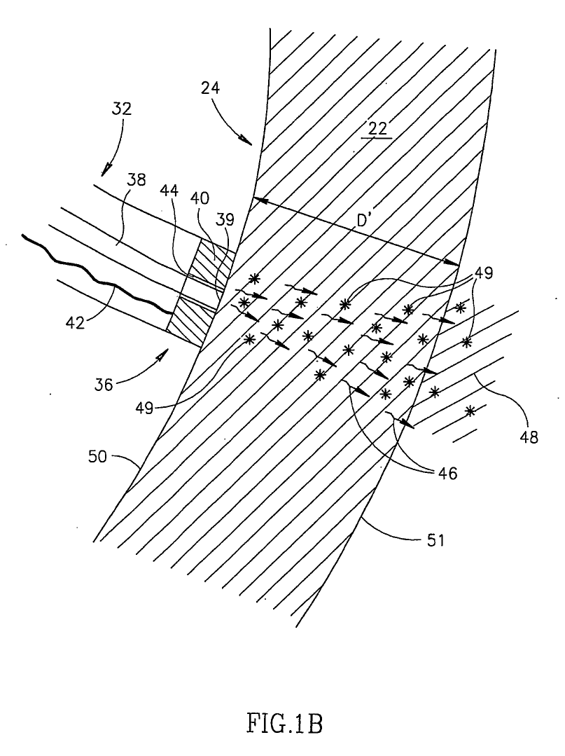

FIG. 1A schematically shows a cutaway view of an MRA 20 configured to perform PMR in accordance with an embodiment of the present invention. MRA 20 is schematically shown performing PMR in a region 22 of the heart wall 24 of the left ventricle 26 of a patient's heart, in accordance with an embodiment of the present invention. MRA 20 comprises a controller 30 and a catheter 32 having a control end 34 coupled to the controller and a drill end 36. Catheter 32 is threaded through the patient's circulatory system and into the left ventricle 26 of the patient's heart so that drill end 36 is optionally in contact with an internal surface 50 of heart wall 24. Any of various methods known in the art may be used to thread drill end 36 into the left ventricle. FIG. 1B shows an enlarged view of region 22 and drill end 36 of catheter 32. Details and features of drill end 36 and region 22 that are not conveniently shown in FIG. 1A on the scale of the patient's heart are shown in FIG. 1B.

By way of example, it is assumed that MRA 20 drills holes in region 22 of heart wall 24 by ablating heart tissue in the region with laser light. Controller 30 provides and controls the laser light and catheter 32 comprises an optic fiber 38 that extends the length of the catheter from control end 34 to drill end 36 for transmitting the laser light from the controller to the region. Controller 30 couples the laser light into optic fiber 38 at an input end (not shown) of the fiber in a neighborhood of control end 34 of catheter 32. The laser light exits the fiber to illuminate region 22 from an output end 39 of the fiber. Drill end 36 of catheter 32 comprises at least one acoustic detector 40 connected to controller 30 via a signal cable 42.

Numerous and varied types of acoustic detectors and arrays of acoustic detectors known in the art may be used in the practice of the present invention. For example, acoustic detector 40 may comprise a single acoustic detector located to one side of fiber 38 or a plurality of acoustic detectors configured in a circular array that surrounds fiber 38. By way of example, in MRA 20 at least one acoustic detector comprises a single annular acoustic detector that optionally, fits snugly in drill end 36 of catheter 32 and is formed with a hole 44 in its center through which optic fiber 38 passes.

In accordance with an embodiment of the present invention, prior to initiating ablation of tissue in region 22, controller 30 transmits at least one pulse of light through optic fiber 38 that illuminates the region with light having an intensity that does not cause ablation but does generate photoacoustic waves in the region. Light from the at least one light pulse, hereinafter referred to as a “mensuration” light pulse, is schematically represented by wavy arrows 46. Intensity and wavelength of light 46 are chosen so that optionally a sufficient amount of light 46 reaches an outside surface 51 of heart wall 24 to generate photoacoustic waves at or close to surface 51 and optionally in tissue in a region 48 beyond surface 51. Photoacoustic waves generated in region 22 responsive to light 46 are represented by starbursts 49 being radiated from “tissue voxels” in the region.

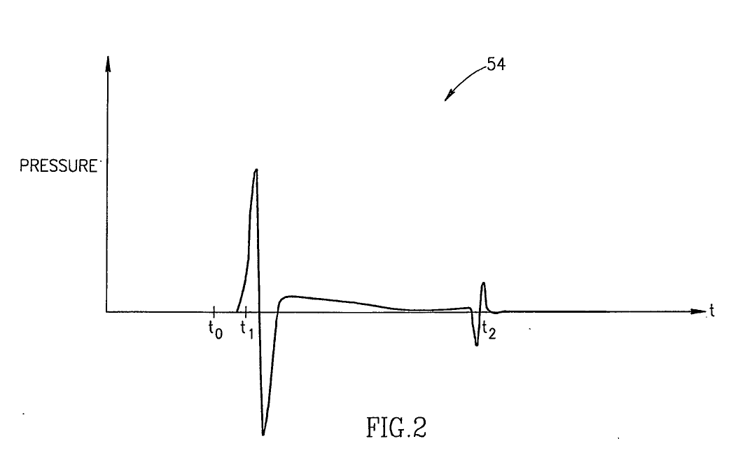

A portion of the acoustic energy in photoacoustic waves 49 is incident on acoustic detector 40, which generates signals responsive to pressure of the incident acoustic energy and transmits the signals via signal cable 42 to controller 30. A schematic graph 54 of amplitude of the pressure of the incident acoustic energy as a function of time following a time tₒ at which light 46 from a mensuration pulse illuminates region 22 is shown in FIG. 2.

Amplitude of pressure in photoacoustic waves generated at a location along the optical path of light 46 is substantially proportional to a first spatial derivative of the energy absorbed from the light per unit volume of material at the location. The pressure amplitude is therefore relatively large and exhibits rapid change at tissue interfaces for which the absorption coefficient of the light changes rapidly. Acoustic energy from photoacoustic waves generated by light 46 is first incident on detector 40, generally with relatively large and rapid changes in pressure, at about a time t₁ from tissue voxels in a neighborhood of inside “interface” surface 50. Time t₁ is substantially coincident with time to because, as is shown in FIG. 1, drill end 36 of catheter 32 and thereby acoustic detector 40 are substantially contiguous with surface 50. A separation of time t₁ from time tₒ is exaggerated in graph 54 for convenience of presentation.

Pressure decreases thereafter until about a time t₂, at which time the pressure again exhibits relatively large and rapid changes as acoustic energy from tissue voxels in a neighborhood of outside “interface” surface 51 reach detector 40. The decrease in pressure between times t₁ and t₂ is a function of an absorption coefficient of tissue in region 22.

In accordance with an embodiment of the present invention, controller 30 processes signals from acoustic detector 40 to identify times t₁ and t₂ using methods known in the art. Controller 30 determines a thickness D (FIG. 1) of heart wall 24 in region 22 by multiplying a difference between times t₁ and t₂ by the speed of sound in cardiac tissue. (In determining D, because the speed of light is so much greater than the speed of sound, a time that it takes light 46 to travel a distance between surfaces 50 and 51 may be neglected.) Optionally, controller 30 performs a plurality of measurements of thickness D to determine the thickness as a function of phase of a heartbeat.

It is noted that to determine D, in accordance with an embodiment of the present invention, it is not necessary that drill end 36 and acoustic detector 40 be contiguous with inside surface 50. For situations in which drill end 36 is not contiguous with inside surface 50, a space between the drill end and inside surface 50 is generally filled with blood. Light 46 from a mensuration pulse transmitted at a time to from output end 39 to determine D stimulates photoacoustic waves in the blood in the space between drill end 36 and inside surface 50 as well as in cardiac tissue in heart wall 24. In particular, a relatively large amount of acoustic energy is generated by the mensuration pulse substantially at time tₒ at the interface between output end 39 and the blood. A time t₁ at which acoustic energy from cardiac tissue in the neighborhood of inside surface 50 reaches acoustic detector 40 follows time tₒ by a delay equal substantially to the distance between drill end 36 and inside surface 50 divided by the speed of sound in blood. Time t₁ and a time t₂ in this situation are identified by relatively large and rapid changes in pressure similarly to the way in which times t₁ and t₂ are identified for the situation in which acoustic detector 40 is contiguous with inside surface 50.

It is also noted that in the above discussion it is assumed that a sufficient portion of light 46 reaches outside surface 51 to generate detectable photoacoustic activity at or near to surface 51. In some embodiments of the present invention, a sufficient quantity of light does not reach surface 51 or tissue close to surface 51 to generate detectable photoacoustic activity at or near surface 51. For such cases, a portion of the photoacoustic energy generated in region 22 propagates to wall 51 and is reflected back to detector 40. A time t′₂ at which the reflected photoacoustic waves reach detector 40 is optionally identified using methods known in the art and used to determine a distance between surfaces 50 and 51.

In some embodiments of the present invention, an MRA similar to MRA 20, comprises at least one external acoustic transducer coupled to the skin of a patient undergoing PMR. The at least one external transducer is used to image the patient's heart during PMR using any of various ultrasound imaging techniques known in the art. In addition, signals generated by the at least one external transducer are optionally used to locate sources of photoacoustic waves in the patient's body generated by light from fiber 38. The location of the sources of the photoacoustic waves may be used to image region 22 and the location of end 39 relative to the region. For example, the photoacoustic waves generated at time tₒ may be used to indicate the interface of end 39 with blood in the heart. For embodiments of the present invention, as discussed below in which tissue is removed by ablative energy that generates acoustic shock waves, the at least one external transducer is optionally also used to determine characteristics of the shock waves and/or locations of their sources.

In some embodiments of the present invention, controller 30 identifies region 22 as an ischemic region appropriate for MR by assaying a component of cardiac tissue 22, whose concentration can be used to determine a degree of ischemia in the region. For example, in some embodiments of the present invention oxygenated hemoglobin in the region is assayed to determine if and to what extent region 22 is ischemic. To assay oxygenated hemoglobin, controller 30 illuminates region 22 with pulses of light at a plurality of different wavelengths, for which for at least one of the wavelengths the light is absorbed by oxygenated hemoglobin, to determine intensity of photoacoustic waves generated at each of the wavelengths. Determined photoacoustic intensities are used to determine a component of the optical absorption coefficient of region 22 due to oxygenated hemoglobin. The component is used to determine a concentration for oxygenated hemoglobin and therefrom an estimate of perfusion of oxygen rich blood in region 22. The estimate of perfusion is used to determine a level of ischemia. In some embodiments of the present invention, drill end 36 of catheter 32 is moved to scan region 22 and assay oxygenated hemoglobin as a function of location in the region and provide thereby an ischemia “map” of the region.

In accordance with an embodiment of the present invention, locations at which angiogenesis holes are drilled in region 22 and characteristics of the holes are optionally determined responsive to the ischemia map. For example, responsive to the ischemia map, an angle at which an angiogenesis hole is drilled into cardiac tissue in region 22 and/or its diameter and/or a density of such holes drilled in the region may be determined responsive to the ischemia map.

In some embodiments of the present invention, concentration of an analyte other than or in addition to oxygenated hemoglobin is used to determine a level of ischemia for region 22. Among analytes that are indicative of ischemia and may be assayed in accordance with an embodiment of the present invention are for example, cytochrome aa₃ redox or Hydrogen ions (corresponding to tissue pH).

In some embodiments of the present invention a rate at which a difference in temperature of tissue in region 22 relative to an “ambient” temperature of heart tissue reverts to the ambient temperature is used to determine a degree of ischemia. A difference in temperature of region 22 or a localized portion of region 22 is produced using any of various methods known in the art. For example catheter 32 may comprise a heating and/or cooling element, such as a suitable Peltier heat pump, located in drill end 36 to heat or cool tissue in region 22. After heating or cooling tissue in region 22 temperature of the tissue is determined as a function of time to provide an estimate of blood flow and thereby ischemia.

In accordance with an embodiment of the present invention, tissue temperature is determined using the photoacoustic effect to measure the absorption coefficient of water in the tissue at at least one wavelength. The measured absorption coefficient and its known dependence on temperature at the at least one wavelength are used to determine temperature of the water and thereby of the tissue. Methods of determining temperature of water and materials comprising water are described in U.S. Provisional Application 60/331,408, and U.S. Pat. No. 6,309,352 the disclosures of which is incorporated herein by reference.

In some embodiments of the present invention, near infrared spectroscopy (NIR) is used to distinguish and identify ischemic regions of heart tissue. Light at a suitable infrared wavelength is transmitted via fiber 38 to illuminate a region of heart tissue. Amounts of light reflected and/or scattered from the transmitted light are detected and used to assay an analyte in the region whose concentration can be used to determine viability of tissue in the region. In some embodiments of the present invention an appropriate optical detector optionally mounted in end 36 of catheter 32 detects the reflected and scattered light. In some embodiments of the present invention; optical fiber 38, and/or additional optical fibers optionally installed in catheter 32, is used to collect the scattered light and pipe the collected light to a suitable detector comprised in controller 30. Various NIR techniques and apparatus known in the art, such as for example those described in U.S. Pat. No. 5,161,531, U.S. Pat. No. 5,127,409 and U.S. Pat. No. 4,967,745, the disclosures of which are incorporated herein by reference, may be used in the practice of the present invention to distinguish and identify ischemic regions of heart tissue.

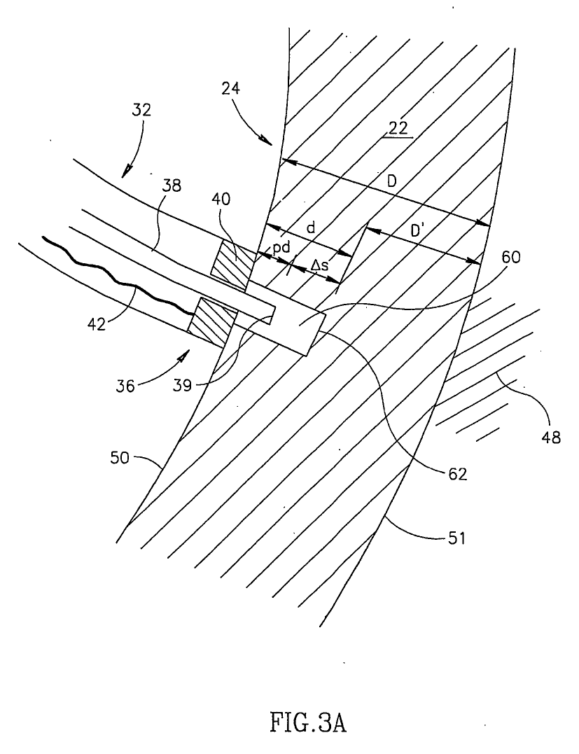

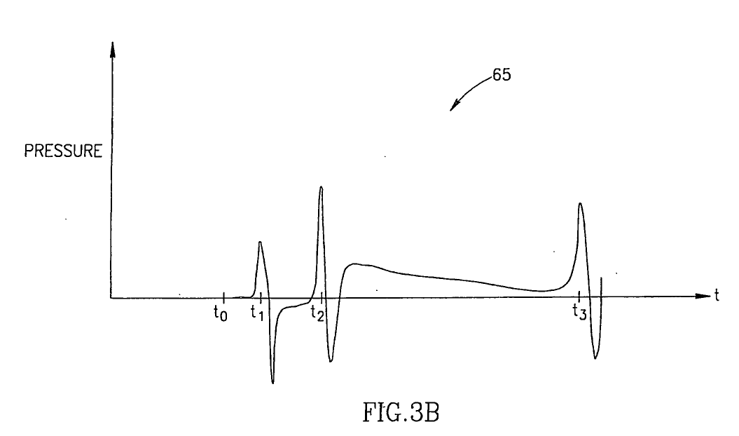

Subsequent to determining thickness D of heart wall 24 in region 22 and/or degree of ischemia in the region, controller 30 transmits relatively intense pulses, “ablation pulses”, of light having an appropriate wavelength via optic fiber 38 to region 22 to ablate tissue in the region and form an angiogenesis hole therein. FIG. 3A schematically shows an enlarged view of region 22 of heart wall 24 after a hole 60 having a bottom 62 has been drilled into the region to a depth “d”.

In accordance with some embodiments of the present invention, as hole 60 is drilled and depth of the hole increases, controller 30 moves output end 39 of optic fiber in the drilling direction so that a substantially constant “separation distance” is maintained between the output end and the bottom of the hole. Controller 30 optionally moves output end 39 of optic fiber 38 by translating optic fiber within catheter 32 so that the output end protrudes beyond drill end 36 by a “protrusion distance” into the hole that is required to provide a desired separation distance. Controller 30 uses any of various methods known in the art, to control motion of optic fiber 38.

In FIG. 3A output end 39 is shown extended beyond drill end 36 by a protrusion distance “pd” so as to provide a desired separation distance “Δs” between the output end and bottom 62. An amount by which to extend output end 39 to provide a desired distance Δs is optionally determined, in accordance with an embodiment of the present invention, as described below.

In some embodiments of the present invention, thickness D′ of tissue between bottom 62 of hole 60 and outside surface 51 is periodically measured to determine depth d of the hole and separation distance Δs. Thickness D′, in accordance with an embodiment of the present invention, is measured similarly to the way in which D is measured, as described above. Controller 30 transmits a mensuration pulse of light 46 (as in FIGS. 1A and 1B) at a time tₒ that illuminates bottom 62 of hole 60 and cardiac tissue between the bottom and outside surface 51. At a time t₁ following time tₒ, acoustic energy reaches detector 40 from photoacoustic waves generated by light 46 in a neighborhood of end 39 of fiber 32, which end as noted above is an interface surface between material in the fiber and blood which fills hole 60. Time t₁, a time t₂ at which photoacoustic energy reaches acoustic detector 40 from cardiac tissue adjacent bottom 62 of hole 60 and a time t₃ at which photoacoustic energy reaches the acoustic detector from cardiac tissue adjacent outside surface 51 are identified. FIG. 3B shows a schematic graph 65 showing amplitudes of pressure sensed by detector 40 from photoacoustic waves originating in neighborhoods of end 39, bottom 62 and outside surface 51 that are used to respectively identify times t₁, t₂ and t₃.

Times tₒ, t₁, t₂ and t₃ may be used in different and various ways to determine geometrical features, such as d, D′, pd and Δs, of hole 60, region 22 and features of catheter 32 relative to the hole, during drilling of the hole. For example, thickness D′ may be determined from t₂, t₃ and the speed of sound in cardiac tissue. Depth d is optionally determined by subtracting thickness D′ from thickness D at a phase of the heart beat at which D′ is determined. Separation distance Δs is optionally determined from times t₁ and t₂ and the speed of sound in blood. Optionally, Δs is determined by subtracting distance pd, from depth d.

Whereas distance pd is, generally, known from an amount by which fiber 38 has been mechanically advanced relative to catheter 32, i.e. by how much the fiber has been pushed into the catheter, pd can also be determined from times tₒ and t₁ and the speed of sound in blood. Alternatively, from a value for pd determined from an amount by which fiber 38 is pushed into catheter 32 and a difference between times tₒ and t₁, the speed of sound in blood can be determined.

It is noted that pd and the size of acoustic detector 40 can be used to determine a time spread of a signal generated by the acoustic detector responsive to acoustic energy that reaches the acoustic detector from a neighborhood of end 39 of fiber 38. The time spread is caused by differences in distances, and thereby of propagation times of sound, between end 39 and different regions of acoustic detector 40. Knowledge of the time spread is optionally used to improve a determination of the speed of sound in blood. Alternatively, knowledge of the time spread as a function of pd may be used to improve accuracy of determination of pd from times tₒ and t₁ and determination of d, D′, or Δs from appropriate functions of times tₒ, t₁, t₂ and t₃.

In some embodiments of the present invention, depth d is determined from a difference between time to and time t₂ and the speed of sound in blood. For example, depth d may be determined from t₂ for situations for which light 46 in a mensuration light pulse is relatively strongly absorbed by cardiac tissue. For such situations light 46 may not generate sufficient detectable photoacoustic activity in cardiac tissue in a neighborhood of outside surface 51 to identify a time t₃. For such situations, as noted above, a time t′₂ at which photoacoustic energy reflected from surface 51 reaches detector 40 is, optionally used to determine D′. In some embodiments of the present invention a wavelength of light that is strongly absorbed by cardiac tissue may purposely be used to illuminate bottom 62 of hole 60 so that photoacoustic waves are generated in a relatively thin layer of cardiac tissue adjacent inside surface 50. Restriction of locations of sources of photoacoustic waves to such a thin layer of tissue can facilitate determination of an accurate value for depth d of hole 60.

In accordance with an embodiment of the present invention, controller 30 controls ablation responsive to measurements of d and/or D′ to drill angiogenesis hole 60 to a desired depth while assuring a sufficient thickness of heart tissue beyond bottom 62 of the hole to prevent perforation of heart wall 24. Controller 30 optionally controls protrusion distance pd responsive to a determined separation distance Δs and a desired separation distance.

In some embodiments of the preset invention, controller 30 automatically terminates ablation when a desired hole depth d and/or tissue thickness D′ is reached. In some embodiments of the present invention controller 30 displays d and/or D′ on a suitable visual display screen and/or alerts an operator of MRA 20 when a predetermined hole depth d and/or tissue thickness D′ is reached and the operator terminates ablation manually.

In some embodiments of the present invention, controller 30 controls ablation of cardiac tissue in region 22 responsive to shock waves that ablation light pulses transmitted by MRA 20 to form hole 60 generate in cardiac tissue in region 22. Each ablation pulse generates an “ablative” acoustic shock wave responsive to a rate at which energy in the pulse removes cardiac tissue. In some embodiments of the present invention, acoustic detector 40 is used to sense ablative shock waves. In some embodiments of the present invention external acoustic detectors (not shown) coupled to the surface of the chest of the patient undergoing ablative MR are used to detect ablative shock waves.

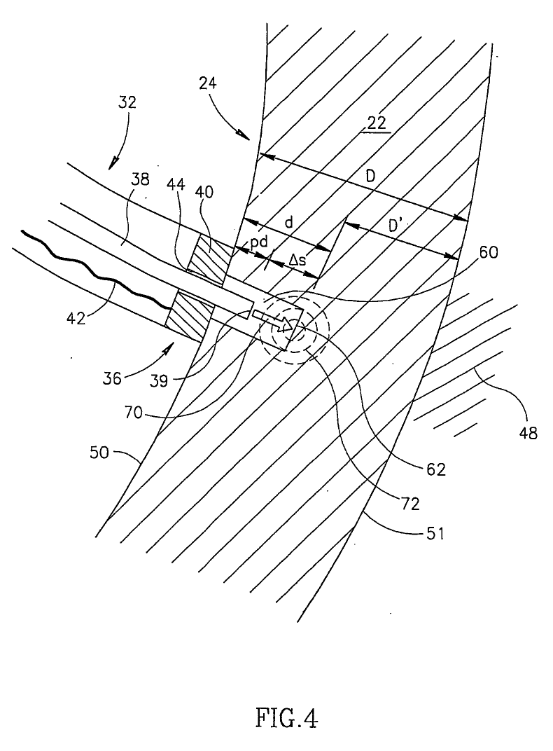

FIG. 4 schematically shows region 22 being illuminated with an ablative optical pulse represented by a block arrow 70 to ablate cardiac tissue from bottom 62 of hole 60. An ablative shock wave generated by ablative pulse 70 is represented by concentric circles 72. In accordance with an embodiment of the present invention, controller 30 controls a characteristic of ablative pulses 70 that MRA 20 transmits responsive to a characteristic of the shock waves. For example, controller 30 optionally controls at least one of pulse width, rise time, fall time, peak, total energy of ablative pulses 70 and wavelength of light in the pulses responsive to a characteristic of the intensity of the shock waves. A characteristic of the shock waves may for example be any one of, or a combination of more than one of temporal profile, maximum, minimum and average pressure, and integrated intensity of the acoustic shock waves.

In some embodiments of the present invention, reflections of acoustic energy from the shock waves 72 are used to determine thickness of the heart wall D or D′ and/or depth d of angiogenesis hole 60. Determined values for D, D′, d are in turn used to control a characteristic of ablative pulses 70 or to determine when to stop ablation.

In some embodiments of the present invention, controller 30 monitors cardiac tissue in region 22 during MR using the photoacoustic effect. In some embodiments of the present invention controller 30 uses the photoacoustic effect to assay a component of cardiac tissue 22 to monitor changes in the tissue generated by the MR procedure. For example, it is expected that drilling angiogenesis hole 60 in region 22 will increase perfusion of oxygen rich blood in the region as blood is forced into angiogenesis hole 60 and therefrom to sinusoids (not shown) in cardiac tissue in the region. Perfusion of blood in region 22 can be assessed during MR, in accordance with an embodiment of the present invention, by assaying oxygenated and/or non-oxygenated hemoglobin or other substances indicative of perfusion in the region using the photoacoustic effect. Assaying is periodically performed similarly to the way in which analytes in tissue region 22 are assayed as described above to determine a degree of ischemia of the region.

In accordance with an embodiment of the present invention, the MR procedure is controlled responsive to the estimate of perfusion. For example, responsive to the perfusion estimate, an angle at which an angiogenesis hole, such as hole 60 is drilled into cardiac tissue in region 22 may be changed or a diameter of angiogenesis hole changed or a density of such holes drilled in the region during the procedure changed.

In some embodiments of the present invention, controller 30 monitors damage to tissue in region 22 that may result from ablative drilling using the photoacoustic effect. For example, it appears that an amount of damage, such as thermal damage, to tissue in a neighborhood of an angiogenesis hole such as hole 60 can be conducive in stimulating angiogenesis a region in which the hole is drilled.

In accordance with an embodiment of the present invention, to monitor possible damage, such as thermal damage that results in denaturing tissue adjacent walls of hole 60, controller 30 periodically illuminates tissue in a neighborhood of hole 60 with mensuration pulses of light that generate photoacoustic waves in the neighborhood. Photoacoustic waves that reach detector 40 are processed to determine whether the received waves indicate damage to the tissue. In some embodiments of the present invention, photoacoustic waves incident on detector 40 generated by mensuration pulses of light 46 that are used to determine depth d of hole 60 are processed to determine damage. In some embodiments of the present invention, a wavelength of light in mensuration pulses used to assess tissue damage is determined so as to increase a difference in the photoacoustic response of damaged tissue relative to undamaged tissue.

In some embodiments of the present invention, an increase in temperature of tissue in a neighborhood of hole 60 is used to monitor and control damage to tissue in the neighborhood. For example, ablation energy is optionally controlled to generate a temperature rise in the neighborhood tissue that causes a desired amount of damage to the tissue. Temperature of tissue in the neighborhood of hole 60 is optionally determined by measuring temperature of water in the neighborhood tissue using a method describe in U.S. Provisional Application 60/331,408 cited above.

In some embodiments of the present invention, a direction along which mensuration pulses illuminate tissue in region 22 is changed to scan the region and “search” for damage. For example, in accordance with an embodiment of the present invention, output end 39 of optic fiber may be directed, using methods known in the art, to illuminate side walls of hole 60 to determine a level of denaturation of tissue along the side walls.

It is noted that whereas the above discussion of examples of embodiments of the present invention relate to PMR, the methods and apparatus, with suitable modifications as might be required and which would readily occur to a person of the art, are applicable to TMR. In addition, whereas in the examples discussed angiogenesis holes are formed by laser ablation, the methods of the present invention apply equally well to forming angiogenesis holes using ablative energy other than laser energy. Finally it is also noted that methods in accordance with an embodiment of the present invention are applicable to forming angiogenesis holes by other than ablation. For example, a method of determining depth of an angiogenesis hole, in accordance with an embodiment of the present invention, may be practiced with substantially any method of forming the hole.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. Apparatus for forming a hole in a region of the heart muscle wall of a patient undergoing myocardial revascularization comprising:
means for removing tissue from the region to form the hole;
a light source that illuminates the region with light that generates photoacoustic waves therein;
at least one acoustic sensor that generates signals responsive to the photoacoustic waves; and
a controller that receives the signals and processes them to determine a characteristic of the region useable to control the means for removing tissue.

2. Apparatus according to claim 1 wherein the light source illuminates the region with at least one pulse of light at a wavelength at which light is absorbed by a substance in the region whose concentration can be used to assess a degree of ischemia in the region and wherein the controller processes the signals provided by the at least one acoustic sensor to assay the substance.

3. Apparatus according to claim 2 wherein the substance is hemoglobin.

4. Apparatus according to claim 3 wherein the hemoglobin is oxygenated.

5. Apparatus according to claim 2 or claim 3 wherein the substance is cytochrome aa₃ redox.

6. Apparatus according to any of claims 1-5 wherein the light source illuminates the region with at least one pulse of light at a wavelength at which light is absorbed by water and determines temperature of the region responsive to the signals.

7. Apparatus according to claim 6 and comprising a heat pump that generates a temperature difference between tissue in the region and an ambient temperature of the heart wall and wherein the controller thereafter determines temperature of the tissue as a function of time tₒ assess a degree of ischemia in the region.

8. Apparatus according to any of the preceding claims wherein the light source illuminates the region with at least one light pulse prior to forming the hole and the controller processes the signals to determine a thickness of the heart wall in the region.

9. Apparatus according to any of the preceding claims wherein after onset of formation of the hole the light source illuminates the region with at least one light pulse that illuminates the bottom of the hole and the controller uses the signals generated by the at least one acoustic sensor to determine a depth for the hole.

10. Apparatus according to claim 9 wherein the controller controls the means for removing tissue from the region responsive to the determined depth and stops formation of the hole by the means for removing tissue when a desired hole depth is reached.

11. Apparatus according to any of the preceding claims wherein the hole is formed in a first surface of the heart wall and deepened towards a second surface of the heart wall and during formation of the hole the light source illuminates the region with at least one light pulse that illuminates the bottom of the hole and the controller uses the signals generated by the at least one acoustic sensor to determine a thickness of the heart muscle wall between the bottom of the hole and the second surface.

12. Apparatus according to claim 11 wherein the first surface is an inner surface of the heart wall.

13. Apparatus according to claim 11 wherein the first surface is an outer surface of the heart wall.

14. Apparatus according to any of claims 9-13 wherein the controller controls the means for removing tissue from the region responsive to the determined thickness and stops formation of the hole by the means for removing tissue when a desired thickness is reached.

15. Apparatus according to any of the preceding claims wherein the means for removing tissue comprises a source of ablative energy having an output port from which the ablative energy source provides energy for removing heart tissue by ablation.

16. Apparatus according to claim 15 wherein the source of ablative energy illuminates the region with at least one pulse of ablative energy to form the hole.

17. Apparatus according to claim 16 wherein the at least one ablative pulse generates an acoustic shock wave in the region responsive to which the at least one acoustic sensor generates signals that are transmitted to the controller and wherein the controller processes the signals to determine at least one characteristic of the shock waves.

18. Apparatus according to claim 17 wherein the controller controls at least one characteristic of the at least one ablative pulse responsive to the determined at least one characteristic of the shock wave.

19. Apparatus according to claim 18 wherein at least one characteristic of the at least one ablative pulse is at least one of pulse width, rise time, fall time, peak, and energy and repetition rate of the at least one ablative pulse.

20. Apparatus according to any of claims 17-19 wherein the at least one characteristic of the shock wave is at least one of temporal profile, duration, maximum pressure, minimum pressure, average pressure average intensity and integrated intensity of the acoustic shock wave.

21. Apparatus according to any of claims 16-19 wherein the pulse generates an acoustic shock wave and wherein an acoustic sensor of the at least one acoustic sensor generates signals responsive to reflections of acoustic energy from the shock wave which the controller processes to determine a characteristic of the region.

22. Apparatus according to claim 21 wherein the characteristic comprises a depth of the hole.

23. Apparatus according to claim 21 or claim 22 wherein the characteristic comprises a thickness of the heart muscle wall between the bottom of the hole and a surface of the wall.

24. Apparatus according to any of claims 16-23 wherein the at least one acoustic sensor generates signals responsive to an acoustic shock wave generated by the at least one ablative pulse and the controller processes the signals to determine location of the source of the shock waves.

25. Apparatus according to any of claims 16-24 wherein the at least one ablative pulse comprises a plurality of ablative pulses.

26. Apparatus according to any of claims 15-24 wherein the light source illuminates the region with at least one pulse of light after onset of ablation and the controller uses signals generated by the at least one acoustic sensor responsive to photoacoustic waves to assess damage to tissue in the region of the hole caused by ablation.

27. Apparatus according to claim 26 wherein the wavelength of the at least one light pulse is determined so as to increase a difference in the photoacoustic response of damaged tissue relative to undamaged tissue.

28. Apparatus according to claim 26 or claim 27 wherein the damage comprises thermal damage.

29. Apparatus according to any of claims 26-28 wherein the damage comprises acidosis.

30. Apparatus according to any of claims 26-29 wherein the controller controls at least one characteristic of the ablative pulses responsive to the determined damage.

31. Apparatus according to any of claims 15-30 wherein the controller processes the signals from the at least one acoustic sensor to determine a distance of the ablative energy output port to the bottom of the hole.

32. Apparatus according to any of claims 15-31 wherein the ablative energy comprises electromagnetic energy.

33. Apparatus according to any of claims 15-32 wherein the ablative energy comprises acoustic energy.

34. Apparatus according to any of claims 15-33 wherein the ablative energy comprises optical energy.

35. Apparatus according to any of claims 15-34 and comprising a catheter having a drill end that is positioned in a neighborhood of or in contact with the region in order to form the hole and wherein the optical output aperture, the ablative energy output port and an acoustic sensor of the at least one acoustic sensor are mounted inside the catheter in a neighborhood of the drill end.

36. Apparatus according to any of claims 15-35 wherein the controller processes signals that it receives from the at least one acoustic sensor to determine a location of the ablative energy output port.

37. Apparatus according to any of claims 1-15 and comprising a catheter having a drill end that is positioned in a neighborhood of or in contact with the region in order to form the hole and wherein the optical output aperture and an acoustic sensor of the at least one acoustic sensor are mounted inside the catheter in a neighborhood of the drill end.

38. Apparatus according to any of claims 35-37 wherein the catheter is configured to perform percutaneous myocardial revascularization.

39. Apparatus according to any of claims 35-37 wherein the catheter is configured to perform transmyocardial revascularization.

40. Apparatus according to any of the preceding claims wherein the at least one acoustic sensor comprises an external acoustic sensor coupled to the patient's skin.

41. Apparatus according to any of claims 1-40 wherein the at least one acoustic sensor comprises an acoustic sensor of an ultrasonic imaging device.

42. Apparatus for forming a hole in a region of the heart muscle wall of a patient undergoing myocardial revascularization comprising:
means for removing tissue from the region to form the hole;
a light source that illuminates the region with light;
an optical sensor that generates signals responsive to light from the light source that is reflected by the region; and
a controller that receives the signals and processes them to determine at least one characteristic of the region useable to control the means for removing tissue.

43. Apparatus according to claim 42 wherein the characteristic is concentration of a substance indicative of viability of tissue in the region.

44. Apparatus according to claim 43 wherein the substance is hemoglobin.

45. Apparatus according to claim 44 wherein the hemoglobin is oxygenated.

46. Apparatus according to claim 43 wherein the substance is cytochrome aa₃ redox.

47. Apparatus according to claim 43 wherein the substance is Hydrogen ions.
